(19) United States
(12) Patent Application Publication
(10) Pub. No.: US 2002/0138053 A1
(43) Pub. Date: Sep. 26, 2002

(54) Absorption of water

(76) Inventors: Ervin Dan, Calgary (CA); Amy Zhong, Calgary (CA)

Correspondence Address: Martin G. Linihan, Hodgson Russ LLP, Suite 2000, One M&T Plaza, Buffalo, NY, 14203-2391, US

(21) Appl. No.: 10/105,996

(22) Filed: Mar. 25, 2002

Related U.S. Application Data
(63) Continuation of application No. 09/051,993, filed on Feb. 26, 1999.
Application No. 09/051,993 is a 371 of international application No. PCT/EP96/04557, filed on Oct. 21, 1996.

(30) Foreign Application Priority Data
Oct. 24, 1995 (GB) 9521779.0
Mar. 4, 1996 (GB) 9604571.1

(51) Int. Cl. A61F 13/15; A61F 13/20

(52) U.S. Cl. 604/367

(57) ABSTRACT

A water-absorbent composition which absorbs water in the presence of multiple valent ions. The water-absorbent composition is a polymeric material having two or more pendent carboxylic acid groups which are in close proximity. The two or more groups react with the ions so that the composition does not precipitate. The pendant carboxylic acid groups can be located on the polymeric backbone in close proximity. The pendant carboxylic acid groups can be located on adjacent carbons on the polymeric backbone. The water-absorbent material can be in fibrous form. The water-absorbent composition can be a copolymer of from about 25 to about 75 mole percent recurring units of at least one k,beta-unsaturated monomer having at least one carboxylic acid group and from about 75 to about 25 mole percent recurring units of at least one copolymerizable monomer.

The present invention relates to the absorption of water containing ions having a multiple valency. More particularly, the present invention relates to the use of a water-absorbent polymeric composition of the kind known as superabsorbent material to absorb water containing multiple valent ions, particularly divalent ions.

As used herein the term “water” when used alone or in phrases such as “water-absorbing”, “water-absorbent” and “water-swellable” is understood to mean not only water but also aqueous media such as, in particular, electrolyte solutions such as body fluids.

A large number of compositions have been developed which exhibit the capacity to be water-absorbing. Known compositions may be in any suitable form including powders, particles and fibers. Examples of two such water-absorbent compositions are described in U.S. Pat. Nos. 3,954,721 and 3,983,095, incorporated herein by reference, which disclose preparations for derivatives of copolymers of maleic anhydride with at least one vinyl monomer in fibrous form. The fibrous copolymers are rendered hydrophillic and water-swellable by reaction with ammonia or an alkali metal hydroxide. U.S. Pat. No. 3 810 468, which is incorporated herein by reference, discloses lightly cross-linked olefin-maleic anhydride copolymers prepared as substantially linear copolymers and then reacted with a diol or a diamine to introduce cross-linking. The resultant lightly cross-linked copolymers are treated with ammonia or an aqueous or alcohol solution of an alkali metal hydroxide. U.S. Pat. No. 3,980,663, which is incorporated herein by reference, describes water-swellable absorbent articles made from carboxylic polyelectrolytes via cross-linking with glycerine diglcidyl ether.

European Published Application No. 0 268 498 (incorporated herein by reference) describes a water-absorbent composition formed by causing a substantially linear polymer of water-soluble ethylenically unsaturated monomer blends comprising carboxylic and hydroxylic monomers to cross-link internally.

Further examples of water-absorbent compositions are those produced from copolymers of an α, β unsaturated monomer having at least one pendant unit selected from a carboxylic acid group and derivatives thereof and a copolymerisable monomer.

A proportion of the pendant units are present in the final copolymer as the free acid and a proportion as the salt of the acid. These copolymers are capable of being cross-linked, either internally or with a variety of cross-linking agents, to form the water-swellable composition. Examples of water-swellable compositions of this type can be found in U.S. Pat. Nos. 4,616,063, 4,705,773, 4,731,067, 4,743,244, 4,788,237, 4,813,945, 4,880,868 and 4,892,533 and EP 0 272 074, 0 264 208 and 0 436 514. These patents and applications are incorporated herein by reference.

Derivatives of carboxylic acid groups include carboxylic acid salt groups, carboxylic acid amide groups, carboxylic acid imide groups, carboxylic acid anhydride groups, carboxylic acid ester groups and the like.

Other examples of water-absorbent compositions can be found in U.S. Pat. No. 4,798,861, WO 93/17066, WO 93/255735, WO 93/24684, WO 93/12275, EP 0 401 044, 0 269 393, 0 326 382, 0 227 305, 0 101 253, 0 213 799, 0 232 121, 0 342 919, 0 233 014, 0 268 498 and 0 397 410, GB 2082614, 2022505, 2270030, 2269602 and 2126591, U.S. Pat. Nos. 4,418,163, 3,989,586, 4,332,917, 4,338,417, 4,420,588 and 4,155,957 and FR 2525121 which are all incorporated herein by reference.

Many of the known water-absorbent compositions having pendant carboxylic acid groups have reduced ability to absorb water which contains multivalent ions, such as divalent ions. Without wishing to be bound by any theory, it is believed that the ions present in the water react with the carboxylic acid groups. As the product of this reaction is insoluble, the absorption of the ions results in the precipitation of the water-absorbent composition which is then no longer capable of absorbing water.

This is a particular problem where the water to be absorbed contains a large proportion of multivalent ions. One example of such water is sea wate.. which contains a number of salts including divalent ionic salts such as MgCl₂, CaCl₂ and SrCl₂. As a typicl use of water-absorbent compositions is as a wrap to protect under sea cables from the effects of sea water, the ability to absorb sea water is particularly important. Multivalent ions can also be found in other “waters” which are generally absorbed by these water-absorbent materials. Examples of the waters include, general spillages, bodily fluids and the like.

We have now discovered that the aforementioned problems can be alleviated or overcame by the use of a water-absorbent composition having pendant carboxylic acid groups arranged in mutual proximity to absorb water containing multivalent, particularly divalent ions. It will be understood that the term “containing” includes the water absorption occurring in the presence of the multivalent ions.

Thus, according to the present invention there is now provided the use of a water-absorbent composition to absorb water containing multivalent ions wherein the water-absorbent composition has pendant carboxylic acid groups and wherein two or more such groups react with the ions and the composition does not precipitate.

The two or more such pendant carboxylic acid groups are preferably located in close proximity. Where the ion to be absorbed is divalent, the pendant carboxylic acid groups are preferably located on the polymeric backbone in close proximity, most preferably, the carboxylic acid groups are located on adjacent carbons on the backbone of the polymer.

Without wishing to be bound by any theory, it is believed that each of the carboxylic acid groups in the two or more pendant groups react with, and complex, the multivalent ion. The resultant composition does not precipitate thereby overcoming the aforementioned problem.

### DETAILED DESCRIPTION OF PREFERRED EMBODIMENTS OF THE INVENTION

Particularly suitable copolymers for use in the production of the water-absorbing compositions used in the present invention will contain from about 25 to about 75 mole percent recurring units of at least one κ,β-unsaturated monomer and from about 75 to about 25 mole percent recurring units of at least one copolymerizable monomer. The copolymer preferably contains from about 35 to about 65 mole percent of recurring units of at least one κ,β-unsaturated monomer and from about 65 to about 35 mole percent of at least one copolymerizable co-monomer. Most preferably, the copolymer will be an equimolar copolymer.

Suitable α,β-unsaturated monomers are those bearing at least one pendant carboxylic acid unit or derivative of a carboxylic acid unit. Derivatives of carboxylic acid units include carboxylic acid salt groups, carboxylic acid amide groups, carboxylic acid imide groups, carboxylic acid anhydride groups and carboxylic acid ester groups.

Examples of suitable α,β-unsaturated monomers include maleic acid, crotonic acid, fumaric acid, mesaconic acid, the sodium salt of maleic acid, the sodium salt of 2-methyl, 2-butene dicarboxylic acid, the sodium salt of itatonic acid, maleamic acid, maleamide, N-phenyl maleimide, maleimide, maleic anhydride, fumaric anhydride; itaconic anhydride, citraconic anhydride; mesaconic anhydride, methyl itaconic anhydride, ethyl maleic anhydride, diethylmaleate, methylmaleate; and the like, and their mixtures. Monomers having two carboxylic acid groups attached to adjacent carbon atoms are particularly preferred.

Any suitable copolymerizable co-monomer can be employed. Examples of suitable copolymerizable co-monomers include ethylene, propylene, isobutylene, C₁ to C₄ alkyl methacrylates, vinyl acetate, methyl vinyl ether, isobutyl vinyl ether, and styrenic compounds having the formula:

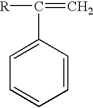

wherein R represents hydrogen or an alkyl group having from 1 to 6 carbon atoms, and wherein the benzene ring may be substituted with low molecular weight alkyl or hydroxyl groups.

Suitable C₁ to C₄ alkyl acrylates include, for example, methyl acrylate, ethyl acrylate, isopropyl acrylate, n-propyl acrylate, n-butyl acrylate, and the like, and their mixtures.

Suitable C₁ to C₄ alkyl methacrylates include, for example, methyl methacrylate, ethyl methacrylate, isopropyl methacrylate, n-propylmethacrylate, n-butyl methacrylate, and the like, and their mixtures.

Suitable styrenic compounds include, for example, styrene, α-methylstyrene, p-methylstyrene, t-butystyrene, and the like, and their mixtures.

The pendant units on the α,β-unsaturated monomer, will determine what, if any, additional reactions must be carried out to obtain a copolymer having the requisite pendant units necessary to produce the water-absorbing compositions of this invention. Preferably these water-absorbing compositions will contain from about 20 to about 80 percent pendant carboxylic acid units and from about 80 to about 20 percent pendant carboxylate salt units. Preferably, both units are present in an amount of from about 30 to about 70 percent.

In general, if the α,β-unsaturated monomer bears only carboxylic acid amide, carboxylic acid limide, carboxylic acid anhydride, carboxylic acid ester groups or mixtures thereof, it will be necessary to convert at least a portion of such carboxylic acid derivative groups to carboxylic acid groups by, for example, a hydrolysis reaction. If the α,β-unsaturated monomer bears only carboxylic acid salt groups, acidification to form carboxylic acid groups will be necessary using methods and materials well known in the art.

Similarly, the final copolymer should contain from about 80 to 20 percent pendant carboxylate salt units. Accordingly, it may be necessary to carry out a neutralization reaction. Neutralization of carboxylic acid groups with a strong organic or inorganic base such as NaOH, KOH, ammonia, ammonia-in-water solution, or organic amines will result in the formation of carboxylate salt units, preferably carboxylate metal salt units.

The sequence and the number of reactions (hydrolysis, acidification, neutralization, etc) carried out to obtain the desired-functionality attached to the copolymer backbone are not critical.

One copolymer particularly suitable for use in the present invention is a copolymer of maleic anhydride and isobutylene. Another is maleic anhydride and styrene. Suitable copolymers will have peak average molecular weights of from about 6,000 to about 500,000 or more.

Suitable copolymers of maleic anhydride and isobutylene can be prepared using any suitable conventional method. Such copolymers are also commercially available from Kurary Isoprene Chemical Company, Ltd., Tokyo, Japan under the trademark ISOBAM. ISOBAM copolymers are available in several grades which are differentiated by average viscosity molecular weight: ISOBAM-10, 160,000 to 170,000; ISOBAM-06, 80,000 to 90,000; ISOBAM-04, 55,000 to 65,000 and ISOBAM-600, 6,000 to 10,000.

The copolymer is then preferably cross-linked either internally via covalent or hydrogen bonding or using an external cross-linking agent. Suitable cross-linking agents include: monomers containing at least two hydroxyl groups such as alkylene glycols containing 2-10 carbon atoms and their ethers, cycloalkylene glycols, Bisphenol A, hydroxy alkylene derivatives of Bisphenol A, hydroquinone, phloroglucinol, hydroxy alkylene derivatives of diphenols, glycerols, erythritol, pentaerythritol, and mono-, di- or oligosaccharides; heterocyclic carbonates; and monomers containing at least one amine group and at least one hydroxyl group such as ethanolamine, tris (hydroxymethyl) aminomethane, 3-amino-1-propanol, DL-1-amino-2-propanol, 2-amino-1-butanol, N,N-dimethylethanolamine, diisopropanol-amine methyl diethanol amine, triethanol amine, 2-(methylamino)ethanol and the like.

In general cross-linking will not occur and the product will not become absorbent until the partially neutralized polymer reaction product is heated to a temperature sufficient to effect reaction between the cross-linking agent and the copolymer.

The cure conditions required to achieve optimal cross-linking depends upon several factors, including the particular polymer employed. For example, the cure temperature will depend on the polymer. If the polymer is a partially neutralized ethylene/maleic anhydride copolymer, a cure temperature of at least 140° C. will be required to achieve cross-linking. If the polymer is partially neutralized styrene/maleic anhydride copolymer, a temperature of at least about 150° C. is required to cross-link; and if a partially neutralized isobutylene/maleic anhydride copolymer is employed, a temperature of at least about 170° C. will be required to achieve cross-linking. Cure times can vary depending on cure temperatures and on the amount of reactive compound used. Cure times will typically be within the range of from about 0.5 to about 20 minutes, preferably 0.5 to 15 minutes, and most preferably 0.5 to 12 minutes. To maximise absorbent properties, optimal cure of the composition (ie. minimal amount of cross-linking needed to form a cross-linked network) is required. Optimal cure is achieved by adjusting a number of variables within wide ranges depending upon the specific syrup composition. Optimal cure conditions require, among other things, a balance between cure time and cure temperature.

As is readily apparent from the high temperature required to achieve cross-linking, the aqueous reaction product of the partially neutralized polymer and the reactive compound, ie. the grafted polymer syrup, can be stored for an unlimited time. This unlimited room temperature stability facilitates further processing of the syrup into any number of conventional forms, including fibers and films using conventional methods. For example, the syrup can be further processed by casting, spray drying, air-assisted spray drying, air attenuation, wet spinning, dry spinning, flash spinning, and the like. To facilitate the removal of water from the aqueous composition during the spinning process, minor amounts of other polar solvents such as alcohol can be added to the aqueous syrups. The resultant fibers can be further processed into milled fibers, chopped fibers, fluff or bulk fibers, strands, yarns, webs, composites, woven fabrics, non-woven fabrics, non-woven mats, tapes, scrim, and the like, using a variety of methods including twisting, beaming, slashing, warping, quilling, severing, crimping, texturizing, weaving, knitting, braiding, etc., and the like.

The water-absorbent compositions may be formed into a composite web comprising non-water absorbent fibers and water-absorbent fibers. The fibers of these webs may be bonded using any suitable technique. Suitable non-water-absorbent fibers include rayon fiber, cellulose ester fiber, protein fiber, polyamide fiber, polyester fiber, polyvinyl fiber, polyolefin fiber, polyurethane fiber, aramid fiber, glass fibers and mixtures thereof.

The composite webs may be used in articles of manufacture such as disposable diapers, sanitary napkins, tampons, pant liners, adult incontinence pads, coverstock for feminine hygiene products, surgical and dental sponges, bandages, patient underpads (for example pairs of the type described in U.S. Pat. Nos. 5,814,101, 4,342,314 and EP 0052403 when are incorporated herein by reference), wipes, domestic wipes, industrial wipes, packaging, filters, medical tray pads, fenestration drapes, operating gowns; mortuary pads, other medical related devices, casket liners, forensic examination pads, cable wrap, food tray pads, food preservation articles, seed germination pads, capillary mats, baby bibs, desiccant strips for anti-rust use, bath mats, sorbents, breast pads, underarm pads, wound covers, pet litter, roofing materials, automotive trim, furniture, bedding, clothing, soil modifiers, spill control materials; waste management materials and protective articles.

These articles of manufacture are found to be particularly effective in absorbing water containing multivalent ions, particularly divalent ions.

Water containing divalent ions include sea water, menses and urine. The composition of synthetic sea water—which is believed to correspond to a typical sea water—is set out in Table 1.

TABLE 1

| Salt | g/l |
|---|---|
| NaCl | 24.54 |
| MgCl₂.6H₂0 | 11.10 |
| Na₂S0₄ | 4.09 |
| CaCl₂ | 1.16 |
| KCl | 0.69 |
| NaHCO₃ | 0.20 |
| KBr | 0.10 |
| H₃B0₃ | 0.03 |
| SrCl₂.6H₂0 | 0.04 |
| NaF | 0.003 |

It can therefore be seen that sea water contains a substantial amount of divalent ions. Therefore using the compositions of the present invention as cable wrap for undersea applications is particularly suitable to protect the cable from the effect of the sea water.

The composition of synthetic adult urine—which is believed to correspond to typical adult urine—is set out in Table 2.

TABLE 2

| Ingredients | g/L |
|---|---|
| Urea | 20.5 |
| NaCl | 8.5 |
| (NH₃)₂ | 2.5 |
| K₂S0₄ | 4.0 |
| Citric acid H₂ | 0.5 |
| MgCl₂.6H₂0 | 1.1 |
| CaCl₂.6H₂0 | 0.8 |
| L. Histidine HCl | 0.7 |
| Albumine bovine | 0.1 |
| Preserving agent | 1.0 |
| Deionised water | 960.3 |
| pH Adjuster | 6.0 |

It can therefore be seen that adult urine contains a substantial amount of divalent ions. Therefore, the compositions of the present invention are particularly useful in, for example, adult incontinence pads.

The following examples are illustrative of the present invention but should not be construed as limiting the invention in any way.

Prepared samples of water-absorbent compositions were tested for water absorbency and fluid retention using the “tea bag” test.

The “tea bag” test was carried out as follows. First 10 pieces of tea bag paper are cut to 5″×2″ and folded to 2.5″×2″ and heat sealed on 2 sides. These bags are then soaked in a test solution, removed, dabbed lightly with filter paper to remove excess saline solution and weighed. These weights are then averaged and the value is recorded as “W₂”. In to a triplicate set of tea bags measuring 5″×2″ is placed approximately 0.2 grams of water-absorbent solvent, the exact weight of which is recorded as “W₃”, the bags loaded with the sample are heat sealed. The triplicate sample containing tea bags are then placed in a test solution, with stirring, for 10 minutes. Each tea bag is then removed from the test solution, allowed to drain for 10 seconds and then dabbed lightly with filter paper to remove excess test solution. Each sample containing tea bag is then weighed and recorded as “W₁”. Each sample containing tea bag is then placed in a Buchner porcelain funnel, a small amount of test solution is poured over the same to re-saturate it, and then the sample containing tea bag is exposed to a vacuum of 0.5 psi for 5 minutes. The sample is removed and weighed and the weight is recorded as “W₄”.

The Free Swell Liquid Retention (FS) is determined using the formula:

g/g=[W₁−W₂)/W₃]−1

The 0.5 psi Liquid Retention (RT) is determined using the formula:

g/g=[W₄−W₂)/W₃]−1

where

W₁=final free swell weight in grams of gel plus wet tea bag,

W₂=average weight in grams of 10 empty, wet tea bags,

W₃=original weight in grams dry staple fiber, and

W₄=final 0.5 psi weight in grams of gel plus wet tea bag.

### EXAMPLES

Commercially available water-absorbent powders A and B were tested in accordance with the above-mentioned test protocols. A water-absorbent composition of the preferred embodiments of the invention were prepared and tested according to the above-mentioned

TABLE 3

| | Composition of | | | | | |
|---|---|---|---|---|---|---|
| | the invention | | Powder A | | Powder B | |
| Test Solution | F.S. | R.T. | F.S. | R.T. | F.S. | R.T. |
| Dionised Water | 351.7 | 249.8 | 278.1 | 190.6 | 209.2 | 148.1 |
| 0.1% CaCl₂ | 117 | 92.4 | 58.5 | 50.1 | 73.6 | 59.3 |
| 0.3% CaCl₂ | 62.1 | 38.6 | 19.3 | 10 | 26.1 | 16.1 |
| 0.5% CaCl₂ | 46.5 | 36.1 | 9.6 | 7.1 | 5.5 | 3.5 |
| 1% CaCl₂ | 43.2 | 27.9 | 3.49 | 2.17 | 10.2 | 5.8 |
| 0.5% Ca(NO₃)₂ | 70.3 | 52.9 | 18.3 | 14.5 | 33.4 | 26.7 |
| 1% Ca(NO₃)₂ | 47 | 34.1 | 6.2 | 5.5 | 7 | 6 |
| 0.5% FeSO₄ | 81 | 56.9 | 33 | 25.4 | 22.4 | 15.8 |
| 1% FeSO₄ | 64.2 | 35.7 | 13.5 | 7.2 | 8.6 | 3.2 |
| 0.1%.Fe₂(SO₄)₃ | 112.7 | 80.2 | 95 | 74.3 | 100.2 | 75.5 |
| 0.5% Fe₂(SO₄)₃ | 43.4 | 24.7 | 40.1 | 30.8 | 38.9 | 29.6 |
| 0.1% MgSO₄ | 122.5 | 87.7 | 85.5 | 61.4 | 69.5 | 50.8 |
| 0.5% MgSO₄ | 66 | 43.4 | 20 | 12.2 | 23.2 | 15.8 |
| 1% MgSO₄ | 55 | 42.7 | 11.9 | 9.3 | 7.5 | 6 |
| Sea Water | 51.4 | 38.4 | 11.3 | 10.2 | 11.2 | 8.7 |
| (synthetic) | | | | | | |
| Sea Water | 57.5 | 43.8 | 19.5 | 16.2 | 14.9 | 12.2 |
| (Vancouver) | | | | | | |
| 1% Urea | 372.8 | 239 | 268.1 | 171.3 | 232.5 | 151.3 |
| 5% Urea | 351.9 | 233.2 | 260.4 | 171.7 | 238.6 | 148.4 |
| 10% Urea | 247 | 177.2 | 230.9 | 172.1 | 215.8 | 151.7 |
| 0.5% ZnSO₄ | 78.5 | 54.2 | 42.1 | 30.3 | 45.5 | 31.6 |
| 1% ZnSO₄ | 67.7 | 50.9 | 20.8 | 15.1 | 20.5 | 13.5 |
| 2% ZnSO₄ | 53.5 | 39.8 | 14.2 | 10.3 | 10.2 | 6 |

1. The use of a water-absorbent composition to absorb water containing multivalent ions characterised in that the water-absorbent composition is a polymeric material having two or more pendant carboxylic acid groups located in close proximity.

2. The use according to claim 1 of a water-absorbent composition to absorb water containing divalent ions characterised in that the water-absorbent composition is a polymeric material having two pendant carboxylic acid groups located in close proximity.

3. The use according to claim 1 or claim 2 wherein the pendant carboxylic acid groups are located on the polymeric backbone in close proximity.

4. The use according to any one of claims 1 to 3 wherein the pendant carboxylic acid groups are located on adjacent carbons on the polymeric backbone.

5. The use according to any one of claims 1 to 4 wherein the water-absorbent material is in fibrous form.

6. The use according to any one of claims 1 to 5 wherein the water-absorbent composition is a copolymer of from about 25 to about 75 mole percent recurring units of at least one κ,β-unsaturated monomer having at least one carboxylic acid group and from about 75 to about 25 mole percent recurring units of at least one copolymerizable monomer.

7. The use according to claim 6 wherein the κ,β-unsaturated monomer is selected from the group consisting of maleic acid, crotonic acid, fumaric acid, mesaconic acid, the sodium salt of maleic acid, the sodium salt of 2-methyl, 2-butene dicarboxylic acid, the sodium salt of itatonic acid, maleamic acid, maleamide, N-phenyl maleimide, maleimide, maleic anhydride, fumaric anhydride; itaconic anhydride, citraconic anhydride; mesaconic anhydride, methyl itaconic anhydride, ethyl maleic anhydride, diethylmaleate, methylmaleate and mixtures thereof.

8. The use according to claim 6 or 7 wherein the co-monomer is selected from the group consisting of ethylene, propylene, isobutylene, C₁ to C₄ alkyl methacrylates, vinyl acetate, methyl vinyl ether, isobutyl vinyl ether, and styrenic compounds having the formula: 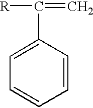
wherein R represents hydrogen or an alkyl group having from 1 to 6 carbon atoms, and wherein the benzene ring may be substituted or unsubstituted.

9. The use according to any one of claims 6 to 8 wherein the copolymer is crosslinked with a crosslinking agent selected from monomers having at least two hydroxyl groups such as alkylene glycols containing 2-10 carbon atoms and their ethers, cycloalkylene glycols, Bisphenol A, hydroxy alkylene derivatives of Bisphenol A, hydroquinone, phloroglucinol, hydroxy alkylene derivatives of diphenols, glycerols, erythritol, pentaerythritol, and mono-, di- or oligosaccharides; heterocyclic carbonates; and monomers containing at least one amine group and at least one hydroxyl group such as ethanolamine, tris (hydroxymethyl) aminomethane, 3-amino-1-propanol, DL-1-amino-2-propanol, 2-amino-1-butanol, N,N-dimethylethanolamine, diisopropanol-amine methyl diethanol amine, triethanol amine and 2-(methylamino)ethanol.

10. The use according to any one of claims 1 to 9 of a water-absorbent composition in an article of manufacture.

11. The use according to claim 10 wherein the article of manufacture is selected from the group consisting of disposable diapers, sanitary napkins, tampons, pant liners, adult incontinence pads, coverstock for feminine hygiene products, surgical and dental sponges, bandages, patient underpads, wipes, domestic wipes, industrial wipes, packaging, filters, medical tray pads, fenestration drapes, operating gowns; mortuary pads, other medical related devices, casket liners, forensic examination pads, cable wrap, food tray pads, food preservation articles, seed germination pads, capillary mats, baby bibs, desiccant strips for anti-rust use, bath mats, sorbents, breast pads, underarm pads, wound covers, pet litter, roofing materials, automotive trim, furniture, bedding, clothing, soil modifiers, spill control materials; waste management materials and protective articles.
